(19) United States
(12) Patent Application Publication
(10) Pub. No.: US 2023/0191195 A1
(43) Pub. Date: Jun. 22, 2023

(54) COMPUTER IMPLEMENTED METHOD AND WEARABLE ELECTRONIC SYSTEM FOR PREDICTING THE OXYGEN UPTAKE DURING AN EXERCISE, AND, NON-TRANSITORY COMPUTER READABLE STORAGE MEDIUM

(71) Applicant: SAMSUNG ELETRÔNICA DA AMAZÔNIA LTDA., CAMPINAS, SÃO PAULO (BR)

(72) Inventors: Marcelo DE OLIVEIRA ROSA PRATES, Campinas (BR); Marcus DE ASSIS ANGELONI, Campinas (BR); Donghyun LEE, Songpa-gy (KR); Intaek OH, Gyeonggi-do (KR)

(73) Assignee: SAMSUNG ELETRÔNICA DA AMAZÔNIA LTDA., CAMPINAS, SÃO PAULO (BR)

(21) Appl. No.: 17/691,353

(22) Filed: Mar. 10, 2022

(30) Foreign Application Priority Data
Dec. 17, 2021 (BR) 10 2021 025549-8

(51) Int. Cl. A63B 24/00; G16H 20/30; G16H 40/67

(52) U.S. Cl. CPC A63B 24/0062; G16H 20/30; G16H 40/67; A63B 2220/836; A63B 2230/75; A63B 2230/06

(57) ABSTRACT

A computer implemented method for predicting the oxygen uptake (% VO₂Max) during an exercise of a user in proportion to the user's maximum oxygen uptake through a wearable device. The method comprising obtaining information from a user profile, capturing at least three temporal input data from a user during an exercise, using the information from a user profile and the at least three temporal input data to extract features to compose features vectors, identifying the exercise being performed by the user based on the feature vectors, selecting a predictor model to compute predictions for the % VO₂Max from the user based on the exercise being performed, and predicting the % VO₂Max from the user based on the selected predictor model.

### CROSS-REFERENCE TO RELATED APPLICATION(S)

This application is based on and claims priority under 35 U.S.C. § 119 to Brazilian Patent Application No. BR10 2021 025549-8, filed on Dec. 17, 2021, in the Brazilian Intellectual Property Office, the disclosure of which is incorporated by reference herein in its entirety.

### FIELD OF THE DISCLOSURE

The present invention describes a method for predicting the oxygen uptake (% VO₂Max) during an exercise of a user in proportion to their maximum by implementing a machine learning model and using a wearable electronic system, such as a wearable device (e.g., smartwatch).

### DESCRIPTION OF RELATED ART

Following the increasing interest by oxygen uptake (VO₂) as a reference standard of exercise intensity, researchers have been developing and recording maximal and submaximal studies aiming to improve the prediction of VO₂ values. These kinds of protocols usually measure gas exchange rates directly through reference equipment and high intensity workloads, rendering the exams costly and physically consuming.

The complexity of direct measures as well as the excessive physical demands exerted on subjects has led to the development of several indirect methods of estimating VO₂ based on submaximal protocols and research focused in predicting VO₂ from submaximal exercise protocols. Such methods differ considerably in a variety of ways. Some employ different submaximal protocols such as ergometer bicycle, aerobic dance, futsal, elliptical trainer, step mill, among others. Using different submaximal protocols can result in different values of VO₂, since each exercise requires a different amount of muscle mass. In addition, many initiatives try to estimate VO₂ by using methods that do not require physical activities.

The actual methods to precisely estimate oxygen uptake (VO₂) and its maximum (VO₂Max) require expensive equipment, exhausting exercise protocols and medical supervision. We propose a method based on machine learning that estimate % VO₂Max on walking or running sessions at the user's own pace, using wearables or other devices with memory constraints.

Maximum oxygen uptake is the maximum rate at which oxygen can be consumed by an individual. It is strongly related to the aerobic metabolism capability of the human body, indicating the maximum ability to synthesize energy in the presence of oxygen. VO₂Max is reached by intense exercises, so any additional work after reaching it can only be done without the use oxygen. Anaerobic breathing fills the gap of energy production, but it produces lactic acid, leading to fatigue. VO₂Max is a common fitness index for athletes, the higher its value, the more conditioned the athlete is. On the other hand, a low VO₂Max value is associated with higher risk of cardiovascular disease. The proportion of the instantaneous (breath-to-breath) oxygen uptake (VO₂) to VO₂Max (VO₂/VO₂Max=% VO₂Max) is not a fitness index in itself but can be used as proxy to estimate the energy expenditure of a physical exercise in real-time.

It is the standard measurement of a person's metabolic rate and subsequently their physical activity as the metabolic equivalent of task (MET), where 1 MET is equivalent to a VO₂ of 3.5 ml/min/Kg. This correlates with the energy expenditure of a physical exercise, such as running or walking. This is relevant for health monitoring, since there have been a number of studies demonstrating that the level of physical activity undertaken by a person is inversely linked with their risk of developing a range of chronic medical illnesses. The % VO₂Max is a necessary component to provide a full health package in wearables.

VO₂ measures the volume of oxygen gas consumed by the body to convert in energy during physical activities. Different activities have different oxygen consumption requirements and increasing the activity intensity results in more oxygen being taken. VO₂Max is the VO₂ reached when oxygen consumption remains at a steady state despite an increase in workload. For running activity, there are two strong predictors of the workload intensity: speed and heart rate. Increasing speed also increases heart rate, as a response for a given stimulus. Depending on the subject fitness, both predictors have characteristic behaviors in time and variation. For example, subjects with better fitness will have higher speeds for longer, heart rate with less variation. Other important predictors include gender, age, weight and height. Speed and heart rate time series with information about the subject compose a high dimensional space where the method searches efficiently for trusted regions to estimate the correct % VO₂Max.

Accurately measuring VO₂Max involves a physical effort sufficient in duration and intensity to tax fully the aerobic energy system. In general, clinical and athletic testing, this usually involves a graded exercise test (either on a treadmill or on a cycle ergometer), in which exercise intensity is progressively increased while measuring ventilation (oxygen and carbon dioxide concentration of the inhaled and exhaled air). VO₂Max is reached when oxygen consumption remains at a steady state despite an increase in workload.

The golden standard methods to measure VO₂ and VO₂Max uses cardiorespiratory devices like Cosmed Quark CPET and Cosmed K5 to acquire gas-exchange data. It involves measurements of respiratory oxygen uptake (VO₂), carbon dioxide expenditure (VCO₂), and pulmonary ventilation during an increased physical workload up to the maximum on a progressive exercise stimulus. In order to accurately measure the VO₂Max value with CPET, the subject must meet some physical criteria while performing the maximum protocol. These criteria are:
minimum duration of the exercise
achieves Maximum Heart Rate (with a tolerance of around 10 beats per minute);
achieves Borg scale for muscular or respiratory effort above 8 (scale 1 to 10);
the VO₂ readings have to indicate the presence of a plateau;
the ratio between the outlet volume of CO2 and the inlet volume of O2 (respiratory exchange ratio) greater than 1.1.

The conventional method of measuring VO₂ and VO₂Max is costly and physically consuming, since the equipment to measure gas exchange is very expensive and the exercise protocol that is needed to reach VO₂Max requires a much higher level of effort than a traditional exercise.

In paper “Estimating Oxygen Consumption from Heart Rate and Heart Rate Variability Without Individual Calibration” by J. Smolander, M. Ajoviita, T. Juuti, A. Nummela & H. Rusko is examined the validity of a method to estimate oxygen consumption (VO₂) based on heart rate, heart rate variability and some features extended of these inputs without any calibration. They evaluated the method in a dataset of 19 subjects performing 25 activities and collecting measures from a set of portable devices. In the present invention, we use a set of inputs available in a single device (smartwatch), the user's profile information (age, gender, height and weight), and the method of the present invention focuses on three specific activities: running, walking, and treadmill exercise. In addition, we evaluated the performance in a dataset with hundreds of subjects with different demographics.

In paper “Instantaneous VO₂ from a Wearable Device” by A. Cook, B. Ng, G. Gargiulo, D. Hindmarsh, M. Pitney, T. Lehmann & T. Hamilton is proposed a method for non-obtrusive calculation of instantaneous oxygen uptake (VO₂). The authors used a single lead electrocardiogram (EG) device combined with a tri-axial accelerometer and some profile data (age and BMI). The ECG was captured at 500 Hz and accelerometer at 120 Hz and some features and regressors were proposed based on these measures. They evaluated the method in a dataset with 42 patients performing a sedentary step, a cardiac stress test on treadmill and an exercise bike test. In the present invention, we use similar inputs, captured by smartwatches and resampled to 1 Hz. The method of the present invention covers exercises performed in the user's own pace (walking, running or a treadmill exercise), and it was validated in a dataset with hundreds of subjects with different demographics. Finally, the method of the present invention can compute the real-time VO₂ for each time window with a pre-defined duration (e.g. 30 seconds) and combine different machine learning approaches with the linear regressor, for example, neural network and extra trees regressor.

In another paper “VO2 Estimation Using 6-axis Motion Sensing Data” by M. Miyatake, N. Nakamura, T. Nagata, A. Yuuki, H. Yomo, T. Kawabata & S. Hara is investigated the accuracy of indirect VO₂ estimation using 6-axis motion sensor (3-axis angular velocity and 3 axis acceleration data) positioned on the waist of the subjects performing a set of exercises with different intensities. In their experiments, conducted in a dataset of 23 subjects, the authors showed that treating data on 6 different axes separately and independently has better estimation accuracy than strategies based on the norm information. Although the series of exercises proposed in this paper is similar to the focus of the present invention, the method of the present invention can use heart rate readings and user profile, both available in the smartwatches, which is known that brings gains in VO₂ prediction. In addition, we perform a light filtering of HR signals based on variance and predefined thresholds tailored for each type of exercise.

The paper “Estimation of Oxygen Uptake During Fast Running Using Accelerometry and Heart Rate” by B. Fudge, J. Wilson, C. Easton, L. Irwin, J. Clark, O. Haddow, B. Kayser & Y. Pitsiladis assessed the feasibility of predict VO₂ from the combined use of accelerometry and heart rate during walking and running. In the experiments of the paper, sixteen endurance-trained males wearing 4 accelerometer devices and a heart rate transmitter performed an exercise protocol composed by walking and running steps. The authors proposed a set of regressor models to predict VO₂ and discussed that the best result covering walking and running was achieved when they combined the tri-axial accelerometer output, heart rate and subject's individual data. In the present invention, it is also focused on walking and running exercises, but the method of the present invention was proposed for use at the population level (not only for endurance-trained males). Since the method of the present invention considers other machine learning models such as extra trees and neural networks, it allows us to find non-linearity by combining the different inputs.

Paper “VO2 Estimation Method Based on Heart Rate Measurement” by Firstbeat Technologies Ltd. describes a method for oxygen consumption (VO₂) developed by Firstbeat Technologies Ltd. Among the four models presented in the whitepaper and validated in a dataset with 32 adults performing submaximal, maximal exercises and real-life tasks, the best result was achieved by a neural network using heart rate, respiration rate (estimated from R-R intervals) and information about it (derived from heart beat data). As the proposition of Firstbeat, the present invention estimate the VO₂ for each time window with a pre-defined duration, but we include in the present invention a prediction of the user' profile information (age, gender, height and weight) as prediction input and the method of the present invention focuses on running and walking exercises. Furthermore, as differences, the present invention employs a combination of machine learning approaches (e.g. a multilayer perceptron neural network, an extra trees regressor and a linear regressor) to estimate % VO₂max and identifies anomalies in the HR, speed and step frequency signals, replacing the unreliable % VO₂max estimations by the most recent non-anomalous estimation.

In patent application “Fitness Test” US 2011/0040193 A1, by Firstbeat describes a method for estimating the maximal oxygen uptake (VO₂Max). The VO₂max estimate is computed from a set of oxygen uptake (VO₂) estimates computed throughout the exercise session, therefore VO₂ is also available as an output. The method uses user profile data variables such as age, weight, height and gender in addition to real-time signals (Heart Rate and Speed). The method includes 1) differentiation between exercise types, 2) segmenting heart rate data to different ranges, 3) calculating the reliability of data segments and 4) calculating weighting coefficients for different data segments. Items 1 and 3 are similar to approaches proposed for the method of the present invention, while items 2 and 5 do not apply. The main innovations in the method of the present invention are the prediction of oxygen uptake (VO₂) in proportion to the maximum (% VO₂max) and its prediction in regular intervals of short duration (e.g. 30 seconds), thus obtaining a denser sequence of estimates in time.

Patent “Exercise Data Device” U.S. Pat. No. 7,643,873B2 by IDT Technology Ltd describes a data apparatus for use on the user's body during exercises (jogging and running), which is composed by an electrocardiogram and a motion detector, enabling the use of heart rate, speed, distance and calorie consumption in its predictions. Among the exercise information computed by the patent, there is the instantaneous VO₂ that is determined for walking and running as linear regression equations using only speed as input. In the present invention, the innovation lies in the method, covering walking and running performed by the users in a treadmill or in any place. The method of the present invention uses more inputs, such as heart rate and profile data, and includes a preprocessing, post-processing step and a combination of machine learning approaches to estimate % VO₂max.

Patent “Device and method for calculating cardiorespiratory Fitness Level and Energy Expenditure of a Living Being” U.S. Ser. No. 10/219,708B2 by Stichting Imec Nederland describes a method for estimating energy expenditure levels from user profile data as well as signals or other estimates available on smartwatches such as heart rate predictions, motion intensity and other accelerometer-related features. The method includes an activity recognition module which is used to assist the energy expenditure prediction based on the estimated activity and a fitness estimation module based on heart rate temporal data and an energy expenditure module which accumulates estimates from other modules as well as user profile data as inputs to compute a final prediction. The energy expenditure estimation is expressed in kcal/min units. The main innovations in the present invention are the proposed techniques for pre and post-processing of the input data and % VO₂max predictions in order to filter out faulty input data and mitigate prediction errors on-the-fly.

The main challenge in this technology is to be equally efficient with very diverse types of people (different genres, ages, body masses). Assembling the best sort of algorithms is the way to enhance this technology such that it covers well most of the demographics. Currently, there is at least one major competitor in the market, Firstbeat, whose technology is in many competitors' products, although its methods focus on VO₂Max estimation as opposed to real-time % VO₂Max estimation.

As can be seen from the state of art, there are several strategies implemented to measure the VO₂Max estimation based on a plurality of exercises made in outdoor or indoor environments using a wearable device. However, the state of art lacks a solution focused on walking and running exercises, which is capable to implement a machine learning model without using a high amount of memory of a device to prediction of oxygen uptake (VO₂) in proportion to the maximum (% VO₂max) in regular intervals of short duration (e.g. 30 seconds), and identifies anomalies in the heart rate, speed and step frequency signals, replacing the unreliable % VO₂Max predictions by the most recent non-anomalous prediction.

### SUMMARY OF THE INVENTION

Considering the aforementioned limitations and difficulties related to a reliable prediction of the proportion of VO₂ in relation to VO₂Max, named % VO₂Max and measured as a percentage (0-100%), to work around this problem, we propose a method to obtain reasonably accurate prediction (i.e. with a mean absolute percentage error lower than 15%) of % VO₂Max, requiring less than 10 KB of memory to run using much cheaper devices than reference laboratorial devices, like smartwatches, and using an exercise protocol with less physical effort.

The present invention, related to the fields of wellbeing, healthcare and artificial intelligence, consists in a technique that predicts the oxygen uptake in proportion to its maximum (% VO₂Max) on walking and running sessions at different paces using wearable devices with memory restriction. Being able to monitor the oxygen uptake is important for good health since it correlates with the level of physical activity of a physical exercise, which in turn is inversely linked to the risk of development of a range of chronic medical illnesses.

In order to achieve this, the present invention proposes a computer implemented method for predicting the oxygen uptake (% VO₂Max) during an exercise of a user in proportion to their maximum through a wearable device, the method comprising the steps of obtaining information from a user profile, capturing at least three temporal input data from a user during an exercise (hearth frequency, speed, and step frequency), using the information from a user profile and the temporal data to extract features to compose features vectors, identifying the exercise being made by the user based on the feature vectors, selecting a predictor model to compute predictions for the % VO₂Max from the user based on the exercise being made and predicting the % VO₂Max from the user based on the selected predictor model.

The method of the present invention was implemented to run on smartwatch microcontroller. Specifically, given a user profile data (age, gender, height and weight) and a set of readings of heart rate (HR), speed and step frequency of a running session at user's own pace, the proposed technique is capable of estimating the % VO₂Max for each time window. The innovative aspects of the present invention include: 1) we perform a light filtering of HR and speed or step frequency signals based on variance and predefined thresholds tailored for each scenario; 2) we employ a combination of machine learning approaches (e.g. a multilayer perceptron, an extra trees regressor and a linear regressor) to estimate % VO₂max from the user profile and input signals and 3) we propose a post-processing step to identify anomalies in the HR, speed and step frequency signals and replace the unreliable % VO₂max estimations by the most recent non-anomalous estimation.

Overall, the main technical advantage over the current state of the art of this method is to achieve good estimations using machine learning and signal processing with restricted memory.

The present invention is also related to a system and a non-transitory computer readable storage medium adapted to performing said method for predicting the oxygen uptake (% VO₂Max) during an exercise of a user in proportion to their maximum through a wearable device.

### BRIEF DESCRIPTION OF THE DRAWINGS

The invention is explained in greater detail below on the basis of figures. Shown therein are:

FIG. 1 shows an overview how the steps of predicting the oxygen uptake (% VO₂Max) during an exercise of a user in proportion to their maximum through a wearable device are performed, according to an embodiment of the present invention.

FIG. 2 shows with more detail the prediction pipeline of the proposed method, according to an embodiment of the present invention.

### DETAILED DESCRIPTION OF THE INVENTION

The present invention aims at providing a new method for predicting the oxygen uptake (% VO₂Max) during an exercise of a user in proportion to their maximum through a wearable device.

For this, we propose a new method to assist users in improving their cardio-respiratory and endurance capacity as well as monitoring their energy expenditure during physical exercises, capable of estimating in real time a fitness measurement known as % VO₂Max without requiring an exhaustive test nor expensive equipment. VO₂ measures the amount of oxygen a person inhales during a single breath, in ml/min/kg.

The proposed method is also useful to monitor exhaustion levels of workers at the workplace (the International Labor Organization suggests a break should be enforced when the worker exceeds a predefined threshold of % VO₂max). This method is not a direct measurement of the value of interest, but an estimation based on a combination of computations over indirect biological signals to extrapolate the value of interest and, as such, requires a refined process to match the operational parameters.

The method uses machine learning to detect patterns to compute predictions, running under restricted memory footprint and using signals that are already available in the wearable (speed, step frequency and heart rate) resulting in a small requirement for execution. Overall, this method is to achieve good estimations using machine learning and signal processing, requiring less than 10 KB of memory.

The machine learning models were trained using a proprietary dataset that allowed understanding correlations between VO₂ information collected by professional equipment and information from wearable sensors.

The machine learning ensemble to estimate % VO₂Max, relying on pattern detection to improve the overall performance in different demographics.

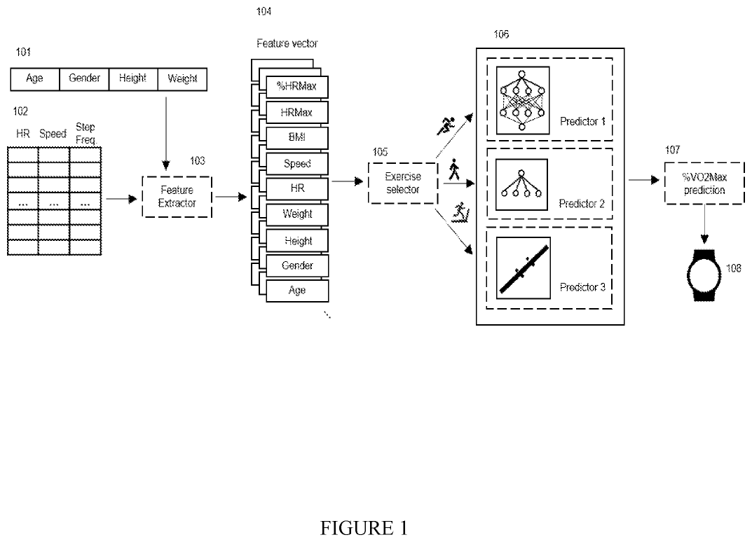

In order to achieve a more precise estimation of % VO₂Max, FIG. 1 shows the computer implemented method proposed by the invention. First, the information from user profile 101, 202 is obtained, which includes age (in years old), gender (male/female), height (in centimeters) and weight (in kilograms). Second, it is captured at least three temporal inputs 102, 201 composed of readings of heart rate, speed (estimated using GPS or wearable device pedometer) and step frequency (estimated from the wearable device accelerometer) during an exercise.

The information obtained from the user profile 101, 202 and the temporal inputs 102, 201 will be used to extract features 103 to composed features vectors 104. The features will be extracted by splitting the temporal inputs 102, 201 in time-windows of N seconds without overlap (e.g. 30 seconds), which are processed by a feature extraction module 103 to obtain a moving average. The feature extraction module is assigned with discarding windows for which the heart rate, speed and step frequency averages fall outside a predefined range, as well as computing new hand-engineered features such as HRMax (the theoretical maximum heart rate, estimated from the user's age), % HRmax (the HR in proportion to HRMax) and BMI (Body-Mass-Index, computed from the user's height and weight).

In order to reduce memory consumption and calculate the mean from the time series, instead of storing M minutes of the signal sampled at 1 Hz we just use the sum of the signal and the sum of the squared signal.

Considering that each temporal data window has N seconds. It is possible to calculate the moving average, defined by:

x̅_{A→A+M} (1)

that is, the mean of the temporal signal with a sliding window of M seconds with a stride of N seconds starting in the second A by using the equation defined by:

x̅_{A→A+M}=x̅_{A→A+N-1}+x̅_{A+N→A+2N-1}+x̅_{A+2N→A+3N-1}+ . . . +x̅_{A+M-N+1→A+M} (2)

Where the mean of the temporal series x̅_{X→Y} from the point X to the point Y is defined by:

$${\overline{x}}_{X \to Y} = \frac{1}{Y - X + 1} {\sum }_{i = X}^{Y} {x}_{i} \qquad ( 3 )$$

by using this representation, it is possible to calculate the moving average just storing M/N variables and obtain a valid temporal data.

Therefore, the feature vectors 104 will be determined by concatenating the profile data 101, 202 with the valid temporal data and the HRMax, % HRMax and BMI data, which will be fed to trained machine learning methods 106.

These feature vectors are used as input for an ensemble composed of three machine learning models, each tailored for a specific scenario and depending on whether the user is walking or running and whether the user is exercising on a treadmill or outdoor 105, 205 a specific method will be used to compute predictions for the user's % VO₂Max 107, 209. For example, but not limited to:

1. A Multi-Layer Perceptron (MLP) regressor for the running scenario.

2. An Extra Trees regressor for the walking scenario.

3. A Ridge regressor for the treadmill scenario.

Finally, the % VO₂Max from the user will be predicted 107, 209 based on the selected predictor model 106 and depending on whether the heart rate deviation 208 exceeds a predetermined ‘instability’ threshold, new prediction 207 or the last stable prediction 206 will be outputted 107, 209.

With the % VO₂Max information in the smartwatch 108, the user can monitor the energy expenditure of a physical exercise in real-time in order to implement improvements and possible adaptations in her or his training. It is an important feedback for the efforts and habits carried out regularly.

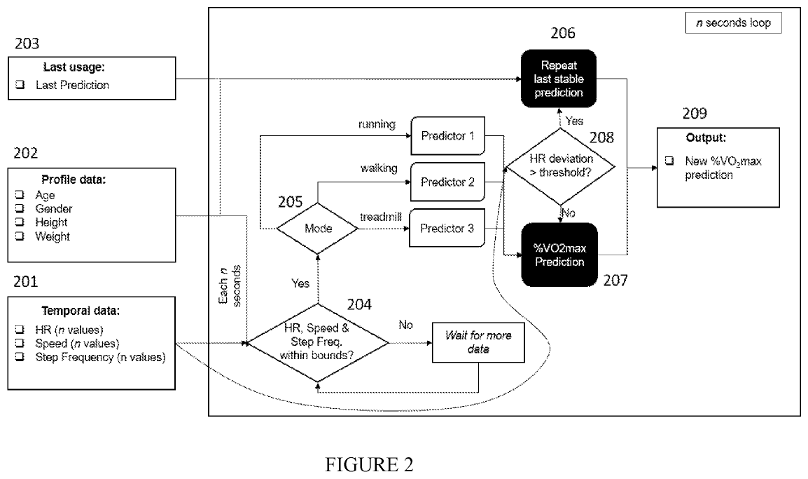

FIG. 2 explains with more detail the prediction pipeline of the proposed method. The diagram illustrates the sequency of a signal processing loop repeated every n seconds. The inputs consist in Temporal data 201 containing sequences of n values for HR, Speed and Step Frequency, Profile data 202 containing Age, Gender, Height and Weight information from the user and the last prediction 203. The first step is to check 204 whether HR, Speed and Step Frequency values are within their respective predetermined bounds and wait for the next window of n values in case not. The model invoked for the estimation (e.g., MLP, Extra Trees or Linear Regression) is dependent on whether the user is running, walking or on a treadmill 205. Finally, there is a post-processing step 208 to decide whether to output the new prediction 207 or to repeat the last stable prediction 206, depending on whether the HR deviation exceeds a predetermined ‘instability’ threshold.

The proposed approach is composed by the fusion of three different models, each fine-tuned to a specific target scenario (running, walking and treadmill exercise). Beyond the model fusion, the approach introduces novel pre and post-processing steps tailored to each scenario to avoid the influence of bad-quality input data and leverage past predictions to mitigate prediction errors in real-time. The individual models were chosen through a comparative analysis of a set of machine learning techniques including linear regression methods, tree, ensemble-based methods and multilayer perceptron-based methods.

The Running model is adapted to the scenario of movement at a running pace in an outdoor environment. In the target scenario, the Speed feature will be computed from the GPS signal of wearable devices, like smartwatches and smartbands. Models are compared based on the simple mean between the SP1 and SP2 Mean Average Percentage Error (MAPE) metric, and the model with the lowest error was chosen.

The Walking model is adapted to the scenario of movement at a walking pace in an outdoor environment. In the target scenario, the Speed feature will be computed from the GPS signal of wearable devices, like smartwatches and smartbands. Models are compared based on walking Mean Average Percentage Error (MAPE) metric, and the model with the lowest error was chosen.

The Treadmill model is adapted to the scenario of movement at a walking to running pace on a treadmill, context in which the GPS speed is unavailable. For that reason, in the target as in the training scenario, the Speed feature is provided by the Step Frequency estimate, which is computed from accelerometer data from of wearable devices, like smartwatches and smartbands, and was registered during the data-collection process, in which volunteers are subject to running or walking protocols on a treadmill. Models are compared based on the simple mean between SP1, SP2 and walking Mean Average Percentage Error (MAPE) metric, and the model with the lowest error was chosen.

Among the potential models, the following can be highlighted:

The Multi-Layer Perceptron (MLP) model consists of a series of fully connected layers of neurons arranged in a feedforward fashion, where each neuron is coupled with a possibly nonlinear activation function. Concretely, the intermediate result of each layer can be expressed by an affine transformation:

A\vec{x}+\vec{b} (4)

over the layer's inputs z, composed with an activation function (in our case the activation function corresponds to:

ReLU(x)=max(0,x) (5)

The series of A and \vec{b} for all layers corresponds to the trainable parameters of this machine learning method.

The hyperparameters of the Multi-Layer Perceptron (MLP) were fine-tuned through an hyperparameter search process, yielding a fully connected neural network with 2 hidden layers of 15 hidden neurons each and ReLU activations on all neurons but the output.

The Extra Trees model computes the % VO₂max prediction as the simple mean of 20 outputs, each computed by a different regression tree. Each tree receives the entire vector of input features z and outputs a prediction. Each leaf node of each tree is associated with a single floating-point number. The tree is traversed from the root node to a leaf through a series of threshold tests on one of the input attributes.

The Ridge model computes the % VO₂max prediction through a simple linear equation:

VO₂Max=−1.005+0.002×Age+0.685×Gender+0.004×Height−0.002×Weight+0.004×HR+0.21×Step Freq.+0.007×BMI−0.001×HRMax+0.008×% HRMax (6)

### Effect

A dataset was collected with high heterogeneity, considering a large variation in terms of user profiles and physical conditions. The data gathering protocol was defined in collaboration with health domain specialists in order to guarantee that the oxygen consumption (VO₂) and heart rate variations could be achieved during the execution of the exercises by the subjects.

All selected subjects had a minimum capacity to perform physical activities and the data gathering sessions happened in an indoor environment.

For stimulating the subjects to achieve different levels of their maximum oxygen consumption and heart rate and allow us to obtain useful data for the method of the present invention, it is defined a data gathering protocol with two steps. The first step is a maximal test using an incremental intensity running to determine the subject's VO₂Max. The second step is a submaximal test to emulate possible regular running conditions.

During the exercises in each protocol, we get the demographic information of the subjects and collected data from wearable devices, including different models of smartwatches and reference equipment.

The collected dataset comprises hundreds of subjects (females and males) with high variability in terms of weight, height, age, skin color and physical conditions.

The innovation of the present invention is a new approach for % VO₂Max estimation based on wearable sensors available in smartwatches, which uses an ensemble of machine learning approaches and introduces new ideas that lead in precise estimations and compact models. The solution of the present invention requires less than 10 KB of memory and was embedded in the microcontroller of smartwatches and smartbands.

### Hardware Implementation

The example embodiments described herein may be implemented using hardware, software or any combination thereof and may be implemented in one or more computer systems or other processing systems. Additionally, one or more of the steps described in the example embodiments herein may be implemented, at least in part, by machines.

Examples of machines that may be useful for performing the operations of the example embodiments herein includes smart bands, smartwatches, fitness trackers, smart clothing, body sensors and other wearable devices.

For instance, one illustrative example system for performing the operations of the embodiments herein may include one or more components, such as one or more microprocessors, for performing the arithmetic and/or logical operations required for program execution, and storage media, such as one or more disk drives or memory cards (e.g., flash memory) for program and data storage, and a random-access memory, for temporary data and program instruction storage.

As is well known in the art, microprocessors can run different operating systems, and can contain different types of software, each type being devoted to a different function, such as handling and managing data/information from a particular source or transforming data/information from one format into another format. The embodiments described herein are not to be construed as being limited for use with any particular type of wearable device, and that any other suitable type of device for facilitating the exchange and storage of information may be employed instead.

Software embodiments of the illustrative example embodiments presented herein may be provided as a computer program product, or software, that may include an article of manufacture on a machine-accessible or non-transitory computer-readable medium (also referred to as “machine-readable medium”) having instructions. The instructions on the machine accessible or machine-readable medium may be used to program a computer system or other electronic device. The machine-readable medium may include, but is not limited to, floppy diskettes, optical disks, CD-ROMs, and magneto-optical disks or other type of media/machine-readable medium suitable for storing or transmitting electronic instructions.

The storage medium comprising computer readable instructions that, when performed by the processor, causes the processor to perform the method steps previously described in this disclosure.

The techniques described herein are not limited to any particular software configuration. They may be applicable in any computing or processing environment. The terms “machine-accessible medium”, “machine-readable medium” and “computer-readable medium” used herein shall include any non-transitory medium that is capable of storing, encoding, or transmitting a sequence of instructions for execution by the machine (e.g., a CPU or other type of processing device) and that cause the machine to perform any one of the methods described herein. Furthermore, it is common in the art to speak of software, in one form or another (e.g., program, procedure, process, application, module, unit, logic, and so on) as taking an action or causing a result. Such expressions are merely a shorthand way of stating that the execution of the software by a processing system causes the processor to perform an action to produce a result.

### Technical Effects Achieved by the Invention

The proposed technique has several advantages for embedded application in wearable devices, such as smartwatches. The method of the present invention can obtain a reasonably accurate prediction (i.e. with a mean absolute error lower than 3 ml/min/Kg) of % VO₂Max using only data and sensors available in most smartwatches, such as profile data, heart rate (HR) based on photoplethysmogram (PPG) sensor, speed computed using accelerometer sensor or Global Positioning System (GPS) libraries and step frequency estimated using accelerometer. Another advantage is that the method does not require maximal exertion on a treadmill or on a cycle ergometer, allowing the user to walk or run at the location of her or his preference and at the pace at which she or he usually trains.

The proposed method is made possible by key innovations to 1) perform on a restricted memory environment; 2) estimate the real-time % VO₂max for each time window with a pre-defined duration (e.g. 30 seconds); 3) be resilient to noise and unreliable data in the input signals and 4) adapt the estimation procedure to the user's target scenario. This is accomplished by developing an ensemble of diverse and lightweight models, each tailored to a particular scenario (running, walking or treadmill) performed in the user's own pace and custom pre and post-processing treatments to filter out noisy or unreliable data before computing predictions as well as a strategy to mitigate the effects of device malfunctions (such as bad contact between the user's skin and the smartwatch).

What is claimed is:

1. A computer implemented method for predicting oxygen uptake (% VO₂Max) during an exercise of a user in proportion to the user's maximum oxygen uptake through a wearable device, the method comprising:
obtaining information from a user profile;
capturing at least three temporal input data from the user during an exercise;
using the information from the user profile and the at least three temporal input data to extract features to compose features vectors,
identifying the exercise being performed by the user based on the feature vectors;
selecting a predictor model to compute predictions for the % VO₂Max from the user based on the exercise being performed, and
predicting the % VO₂Max from the user based on the selected predictor model.

2. The computer implemented method according to claim 1, wherein the obtaining information from user profile comprises collecting at least user data like age, gender, height and weight.

3. The computer implemented method according to claim 1, wherein the capturing at least three temporal input data from the user profile comprises:
collecting readings of at least heart rate, speed, or step frequency.

4. The computer implemented method according claim 1, wherein the using the information from the user profile and the at least three temporal input data to determine the features vectors comprises:
splitting readings of the at least three temporal input data in time windows of N seconds without overlap to obtain a moving average,
discarding temporal data that falls outside a predetermined range to obtain valid temporal data, wherein the predetermined range comprises threshold values based on the moving average of the temporal data, and
associating the user profile data with valid temporal data to calculate maximum heart rate (HRMax), estimated heart rate (% HRMax) and Body-Mass-Index (BMI) from the user.

5. The computer implemented method according to claim 1, wherein readings of the at least three temporal input data are sampled at 1 Hz and split into time windows of 30 seconds.

6. The computer implemented method according to claim 1, wherein the at least three temporal input data comprises a temporal signal and a temporal series,
wherein a moving average of the temporal signal with a sliding window of M seconds with a stride of N seconds starting in the second A is calculated by using the following equation:
x̅_{A→A+M}=x̅_{A→A+N-1}+x̅_{A+N→A+2N-1}+x̅_{A+2N→A+3N-1}+ . . . +x̅_{A+M-N+1→A+M}
wherein the moving average of the temporal series from the point X to the point Y is calculated by using the following equation:
$${\overline{x}}_{X \to Y} = \frac{1}{Y - X + 1} {\sum }_{i = X}^{Y} {x}_{i}$$

7. The computer implemented method according to claim 1, wherein the determining the features vectors is carried by:
concatenating the user profile data, valid temporal data and the HRMax, % HRMax and BMI data.

8. A method according to claim 1, wherein the identifying the exercise performed by the user comprises:
detecting whether a type of exercise is at least one of the scenarios: running scenario, walking scenario or treadmill scenario.

9. The computer implemented method according to claim 1, wherein the selecting the predictor model to compute predictions for the % VO₂Max from the user comprises:
choosing at least one machine learning model among a Multi-Layer Perceptron (MLP) regressor for the running scenario, an Extra Trees Regressor for the walking scenario and a Ridge Regressor for the treadmill scenario.

10. The computer implemented method according to claim 1, wherein predicting the % VO₂Max from the user, comprises:
associating the feature vectors with the predictor model to determine the % VO₂Max from the user,
wherein
a new prediction is output provided a heart rate deviation does not exceed a predetermined instability threshold, or
a last stable prediction is output provided the heart rate deviation exceeds a predetermined instability threshold.

11. A wearable electronic system for predicting the oxygen uptake (% VO₂Max) during an exercise of a user in proportion to the user's maximum oxygen uptake through a wearable device, comprising:
a processor;
a memory including computer readable instructions that, when executed by the processor, causes the processor to perform the method as defined in claim 1.

12. The wearable electronic system according to claim 11, wherein the wearable electronic system is a smartwatch, smart band, fitness tracker, smart clothing or body sensors.

13. A non-transitory computer readable storage medium, which stores computer readable instructions which, when executed by a processor, causes the processor to perform the method as defined claim 1.
